(19) United States
(12) Patent Application Publication
(10) Pub. No.: US 2002/0161347 A1
(43) Pub. Date: Oct. 31, 2002

(54) Absorbent article

(76) Inventors: Arie Besemer, Amerongen (NL); Anne-Mieke Verwilligen, Zeist (NL)

Correspondence Address: Ronald L. Grudziecki, BURNS, DOANE,, SWECKER & MATHIS, L.L.P., P.O. Box 1404, Alexandria, VA, 22313-1404, US

(21) Appl. No.: 10/071,082

(22) Filed: Feb. 11, 2002

Related U.S. Application Data
(60) Provisional application No. 60/268,679, filed on Feb. 15, 2001.

(51) Int. Cl. A61F 13/15

(52) U.S. Cl. 604/367

(57) ABSTRACT

The invention relates to a disposable absorbent article to be worn by a user comprising an absorbent body (104) and a cover enclosing the same, which on a first side, facing the user in an in-use position, displays a fluid-pervious surface layer (102) and on a second side, facing away from the user in an in-use position, displays a fluid-impervious surface layer (103), and is characterized in that the fluid-pervious surface layer (102) comprises an impregnation comprising at least one hydrophilic organic solvent having a low vapor pressure at room temperature.

### TECHNICAL FIELD

The present invention relates to a disposable absorbent article to be worn by a user comprising an absorbent body and a cover enclosing the same, which on a first side, facing the user in an in-use position, displays a fluid-pervious surface layer and on a second side, facing away from the user in an in-use position, displays a fluid-impervious surface layer.

### BACKGROUND ART

Certain types of absorbent articles such as sanitary napkins for female users are intended to be worn in close contact with the body of the user. Such an absorbent article is thereby usually applied inside the panties of the user and is kept in contact against the body by the pressure from the panties during use.

It is important that the surface of an absorbent article which is intended to be facing the body of the user is soft and comfortable and does not cause irritation. Furthermore, the surface of the article should have the ability to receive the body fluid which is emitted to the article, and rapidly let the fluid pass into the article and be absorbed by it. If the fluid is not admitted into the article sufficiently quickly, the risk is obvious that the fluid will instead flow on the surface of the article and cause leakage. Furthermore, the surface of the article will become wet and sticky, something which is perceived as extremely unpleasant by a majority of users. A wet surface may further cause the user inconvenience in the form of skin irritation.

In order to avoid wet surfaces on absorbent articles, these are generally provided with a fluid-pervious surface layer of a comparatively hydrophobic and non-absorbent material. Examples of such hydrophobic non-absorbent materials are perforated plastic films, plastic scrims and nonwoven materials of hydrophobic fibres. The term nonwoven material refers to different types of nonwoven, bonded fibre layers. Such hydrophobic layers have a very low wettability and ability to admit fluid there through. For this reason, the acquisition rate of hydrophobic surface materials is often too low.

The hydrophobic surface layers exhibit a very dry surface, even after wetting. However, small fluid quantities may remain in or on the surface layer after wetting, since the fluid transportation ability in a hydrophobic surface layer is low. It is true, for instance, that a perforated plastic layer has a good fluid transportaion ability through the perforations, but fluid which has ended up between the perforations tends to remain on the surface.

Remaining fluid in or on the fluid-pervious surface layer constitutes a problem, in particular when the absorbent article is a sanitary napkin, since menstrual fluid and blood have a relatively high viscosity and thereby has a larger inclination to leave residues on the fluid-pervious surface layer. Such remaining fluid results in the surface of the sanitary napkin becoming wet and sticky, which is a disadvantage both during use and when the sanitary napkin shall be replaced. Moreover menstrual fluid on the fluid-pervious surface layer is often taken as an indication that the article is saturated with fluid and should be changed. Thus, many absorbent articles are discarded long before they have been fully utilised. In addition, fluid remaining on the outside of the absorbent article is perceived as unhygienic, and gives a feeling of uncleanness.

A number of different types of treatment, chemical or physical, for modifying the surface layer and changing the surface properties of materials for absorption purposes are well-known to the person skilled in the art. The most common way to create hydrophilicity for an initially hydrophobic material is to treat the material with a wetting agent. The purpose of this treatment is to achieve an improved wettability and thereby improved permeability of fluids.

As an example, the document WO 98/03716 discloses a process for providing fibres or nonwovens with a hydrophilic coating. The fibres and nonwovens are treated with an aqueous dispersion of a preparation consisting of a monoester of glycerol and a C₆-C₁₄ fatty acid. According to WO 98/03716 treated nonwovens of the above-mentioned type display “liquid strike through” times of less then 10 seconds for synthetic urine (0.9% NaCl), measured according to EDANA test method 150.0-8.

As is a well known fact in the art, real blood and menstrual fluids differ considerably and in many respects from urine, for example as to surface tension, viscosity (12 centipoise and 1 centipoise for blood and urine, respectively) and presence of particles. Consequently, blood absorption differs in many aspects from urine absorption.

Thus, there is a need for a surface material displaying enhanced absorption rate to fluids like menses and real blood. Further, to reduce the costs of production, it is desirable to find a preparation comprising a less complex compound than a monoester from glycerol and a fatty acid as disclosed in WO 98/03716.

A further drawback with a preparation according to WO 98/03716 is that the technical solution necessitates the application of several production steps in the manufacture of the hydrophilic nonwovens. Hence, the nonwoven is brought into contact with the preparation which is in the form of a dispersion, whereafter the treated nonwovens are passed through a pair of rollers for dewatering of the fibres, and subsequently, a final step, wherein the fibres are dried.

Accordingly, in order to reduce the costs of production for a hydrophilic surface material, the number of steps in the manufacturing process of the hydrophilic surface material needs to be reduced. Especially, there is a need to eliminate the drying step, as drying is a particularly expensive and energy-consuming procedure and contributes significantly to the costs of production.

### DISCLOSURE OF INVENTION

In accordance with the present invention an absorbent article has been provided, which article eliminates the problems set out above.

An object of the present invention is to provide an absorbent article exhibiting an enhanced absorption rate of menstrual fluid and real blood. This object is achieved in accordance with the present invention with an article of the kind mentioned in the introduction and characterized in that the fluid-pervious surface layer comprises an impregnation comprising a hydrophilic organic solvent having a low vapour pressure at room temperature. Preferably, the hydrophilic organic solvent has a vapour pressure less or equal to 1 mm Hg at 40° C.

The invention is particularly useful for absorbent articles such as sanitary napkins and panty liners.

It is also possible to use a hydrophilic organic solvent having a boiling point of at least 150° C.

In one embodiment of the present invention, the hydrophilic organic solvent preferably has a high oxygen content, preferably at least 30%.

According to one embodiment of the invention the hydrophilic organic solvent is selected from the group of alcohols, ethers or polyether alcohols, or a mixture thereof.

According to another embodiment of the invention the hydrophilic organic solvent is glycerol. Other possible hydrophilic organic solvents to use are for example, ethylene glycol (3844) and/or glycol ethers, preferably polyethylene glycol (7729), 2-ethoxyethanol (3797), 1,1′-oxybis(2-ethoxy)ethane (3167), 1,1′-oxybis[2-methoxyethane] (3208), 2,2′-oxybisethanol (3168), 2-methyl-2,4-pentane diol, propylene glycol (8040), triethylene glycol (9802), 1,3-butylene glycol (1601), 2,3-butylene glycol (1602), or tetraglyme (9348), or mixtures thereof.

The figures mentioned in parenthesis are the numbers appearing in the book The Merck Index, 12^{th} edition, Whithous Station, N.J. (1996), Susan Budavari (Ed.).

The compounds listed above are soluble in water and most of the compounds are infinitely miscible with water.

Accordingly, it is possible to use an impregnation which comprises water and at least one hydrophilic organic solvent of the above-mentioned kind. Such an impregnation has the advantage of being easy to apply as it exhibits a low viscosity due to the addition of water. However, the amount of water is so small that no drying step is needed, i.e. the amount of water is so small that it will not affect the absorption properties of the absorbent article.

Some of the compounds mentioned above have a low boiling point but will afford good performance when mixed with glycerol. However, from a practical point of view and considering safety and occupational hazards, it is preferable if low vapour pressure materials are used.

It is preferable to apply the hydrophilic organic solvent through spray nozzles as the amount of the impregnation hereby is easy to adjust. Naturally, any suitable application method can be used such as roller coating, printing, dip coating etc., which gives for the purpose a suitable spreading image.

After application, the hydrophilic organic solvent is found in the pores of the fluid-pervious surface layer. During use of the article the hydrophilic organic solvent will gradually be transferred to the core by the absorbed menstrual fluid or real blood. Thus, the hydrophilic organic solvent will not be in contact with the skin of the user during use of the article.

According to one embodiment of the invention the impregnation further comprises an acid. Preferably, the acid is non-volatile since most volatile acids have bad smells. The acid will have an effect on the pH control with the purpose of inhibiting growth of unwanted micro-organisms to counteract bad smells or irritation of the skin or mucous membranes in the urogenital region of the user. The inhibition effect is based on the fact that many micro-organisms have an activity which is strongly pH-dependent and decreases with decreasing pH, which means that a decrease in pH leads to a decrease in activity of most micro-organisms which, in turn, leads to a decrease of bad smell as well as negative effects on skin in the form of skin-irritation and primary or secondary skin-infections and a generally lower risk of infections.

The acid is preferably a hydroxy acid and most preferably lactic acid. Another suitable acid is tartaric acid, which can be applied in powder form, by sprinkling or by any other well known application method.

In order to effect a good pH control (preferably in the range from 4 to 5) at least 200 mg of lactic acid should be added to the impregnation, based on an expected release of 15 ml of menstrual fluid.

An absorbent article according to the present invention, which displays an enhanced absorption rate to menstrual fluid and real blood in relation to prior art, and which comprises an initially hydrophobic surface layer which has been impregnated with a hydrophilic compound, is in comparison with prior art, also cheaper to manufacture as the impregnation comprises an inexpensive compound, such as for example, glycerol.

Further, an absorbent article according to the present invention is manufactured without the steps of dewatering and drying after the impregnation has been applied on the initially hydrophobic surface material. Thus, an absorbent article according to the present invention, is in comparison with prior art, even cheaper to manufacture as an article according to the present invention does not need a dewatering step nor an expensive and energy-consuming drying step.

### BRIEF DESCRIPTION OF DRAWINGS

The invention will now be described in greater detail with reference to the illustrative embodiments represented in the attached drawings, in which:

FIG. 1 shows a plan view of a sanitary napkin, seen from the side intended to be directed towards the user when the sanitary napkin is in use;

FIG. 2 shows a cross section along line II-II through the sanitary napkin in FIG. 1.

### DETAILED DESCRIPTION OF THE EMBODIMENTS

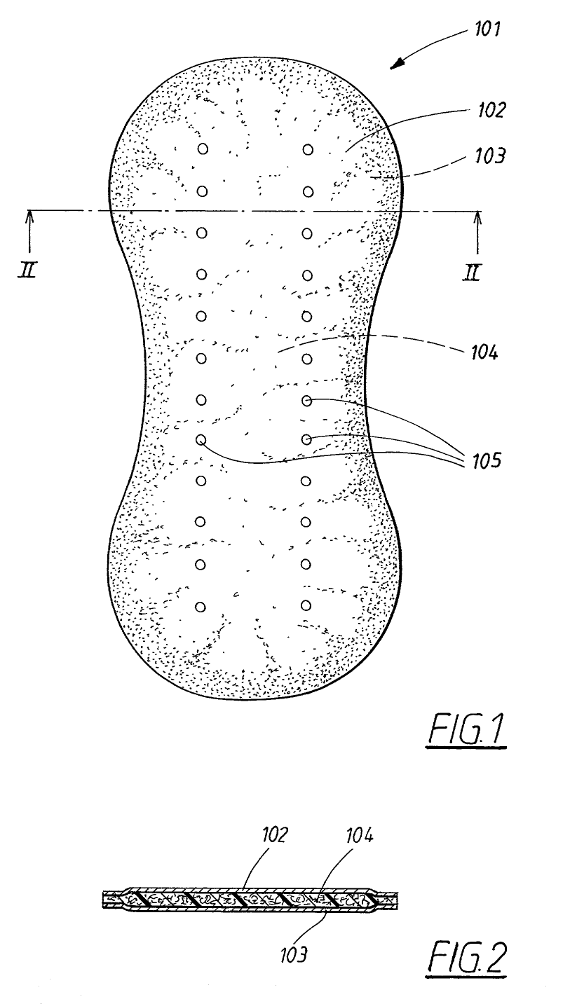

In FIG. 1 a sanitary napkin 101 is shown, comprising a fluid-pervious surface layer 102, a fluid-impervious surface layer 103 and an absorbent core 104 sandwiched therebetween. The fluid-pervious surface layer 102 is arranged on that side of the sanitary napkin 101 which during use is intended to be directed towards a user, and the fluid-impervious surface layer 103 is arranged on that side of the sanitary napkin 101 which during use is intended to be directed away from the user.

FIG. 2 shows a cross section through the sanitary napkin 101 along the line II-II. The fluid-pervious surface layer 102 is of a conventional type and can therefore consist of any fluid-pervious material suitable for the purpose. Examples of such materials are different types of thin nonwoven material, perforated plastic films, net material, fluid-permeable foam material or the like. The fluid-pervious surface layer 102 can be constructed from two or more different materials in order to provide different functions of the surface layer. For example, it is usual to arrange a fluid-transporting layer inside of a fluid-admission layer. It is also known to arrange different types of material on different parts of that surface on the sanitary napkin which faces the wearer during use. A material with good admission capacity can therefore advantageously be arranged in that portion of the sanitary napkin which is expected to be wetted first by the major part of the bodily fluid, while portions of the surface layer, which are primarily intended to constitute a contact surface against the body of the wearer are provided with a material which has been optimized with respect to softness and kindness to the skin.

It is not necessary for the invention that the fluid-pervious surface layer 102 consists of a separate material layer, but the surface layer 102 can be a surface on the absorption body 104 of the sanitary napkin 101. However, in such an embodiment, it is particularly advantageous to provide the sanitary napkin 101 with some form of fluid barrier which prevents fluid from being carried in the absorption material right out to the edges of the sanitary napkin 101. Examples of such fluid barriers are compressions, welds, adhesive bands, folded-back plastic strips or hydrophobicizing means such as wax or the like.

The fluid-impervious surface layer 103 can consist of any suitable fluid-tight material. Particularly advantageous materials are thin plastic films, fluid-tight nonwoven materials, or materials which are coated with fluid-tight material such as wax, resin, adhesive or the like. It is also possible to use fluid-tight material laminates. It may be desirable, for example, to provide the rear side of the product with an outer layer of a textile nature, for example a nonwoven layer. Such a nonwoven material provides a soft skin-friendly textile surface and affords advantages such as a high degree of wearer comfort, high friction and thus better retention in the underwear. Furthermore, a textile surface is often considered to have an aesthetically attractive appearance. It is also an advantage if the fluid-impervious surface layer 103 is breathable, that is to say it allows gas and water vapour to pass through the layer.

The absorption body 104 can be an airlaid cellulose body or can be made up of any suitable absorbent material.

Other suitable absorbent materials for use in the absorption body 104 are, for example, cellulose fluff pulp, absorbent bonded fibre layers, tissue layers, absorbent foam, peat or the like. The absorption body 104 can also contain superabsorbent polymers, i.e. polymers which are able to absorb several times their own weight of fluid and form a fluid gel. Superabsorbents are generally present in the form of particles, flakes, fibres, granules or the like. The superabsorbent material can be used alone or in combination with other absorbent material.

It is not necessary for the invention that the surface layers 102, 103 and the absorption body 104 to have the same extent in the plane of the product, cf. FIG. 2. Hence, it is alternatively possible to enclose the absorption body in a conventional manner between two surface layers with somewhat greater extent in the plane than the absorption body. In such an embodiment, the surface layers are interconnected within an edge join projecting around the absorption body. Such an edge join can be produced by, for example, gluing, sewing or welding using heat or ultrasound.

The invention will now be described in more detail by way of the following non-limiting examples. In the examples defibrinated sheep blood was used as a representative for menstrual fluid and real blood.

### EXAMPLE 1

In order to create a hydrophilic wettable surface on the fluid-pervious surface layer 102 of the sanitary napkin 101 an impregnation was applied on the fluid-pervious surface layer 102, on that side which will face the user in an in-use position of the sanitary napkin 101. The impregnation was applied by evenly distributing droplets of the impregnation on the fluid-pervious surface layer 102. The size of the droplets was in the range from 10 to 20 μl. The droplets were then evenly spread out on the fluid-pervious surface layer 102 by means of a spatula. The means of spreading of the droplets is not critical. Hence, other ways of spreading the droplets can be used, such as a rubber roll, or the like.

Droplets of sheep blood 105 were then applied on the impregnated fluid-pervious surface layer 102 as shown in FIG. 1. The absorption rate was then measured. Here, the absorption rate is defined as the number of blood droplets 105 absorbed on the sanitary napkin 101, i.e. the number of blood droplets 105 passing through the impregnated fluid-pervious surface layer 102 to the underlying absorption body 104. The absorption rate, or the numbers of blood droplets 105, were measured 10, 120 and 600 seconds after the application of the blood droplets 105 on the fluid-pervious surface layer 102. The absorption rate was not measured for longer periods of time as blood droplets 105 residing on the fluid-pervious surface layer 102 change their properties, probably due to evaporation of water, which makes measurements for longer times meaningless.

The following table summarises the results obtained for various impregnations wherein the impregnation may comprise one or two hydrophilic organic solvents. In one of the tests the impregnation was mixed with water.

The table also include absorption rates measured for non-treated fluid-pervious surface layers 102, i.e. fluid-pervious surface layers 102 that have not been impregnated with a hydrophilic organic solvent.

From the table, it can be established that the absorption rates for the impregnated sanitary napkins are superior to those obtained for the un-impregnated sanitary napkins. Hence, an absorbent article according to the present invention exhibits an enhanced absorption rate of menstrual fluids and real blood in comparison to an untreated absorbent article.

TABLE 1
Blood absorption rate on impregnated sanitary napkins.

| Solvent(s) | N¹⁾₀ | N²⁾₁₀ | N³⁾₁₂₀ | N⁴⁾₆₀₀ |
|---|---|---|---|---|
| None | 21 | 1 | 2 | 3 |
| None | 22 | 2 | 2 | 2 |
| None | 25 | 2 | 3 | 4 |
| PEG⁵⁾(400) | 24 | 12 | 17 | 20 |
| PEG⁵⁾(400) | 24 | 17 | 19 | 22 |
| PEG⁵⁾(400) | 22 | 15 | 17 | 21 |
| Glycerol/PEG⁵⁾(400)⁶⁾ | 22 | 20 | 21 | 21 |
| Glycerol/PEG⁵⁾(400)/Water⁷⁾ | 24 | 20 | 21 | 22 |
| 1,4-butanediol | 22 | 13 | 15 | 18 |
| 1,4-butanediol | 24 | 18 | 19 | 21 |
| Glycerol | 24 | 21 | 22 | 23 |
| 1,4-butanediol/PEG⁵⁾(400)⁸⁾ | 30 | 21 | 25 | 28 |

¹⁾Number of droplets applied on the sanitary napkin 101 at t = 0 seconds.
²⁾Number of droplets absorbed at t = 10 seconds.
³⁾Number of droplets absorbed at t = 120 seconds.
⁴⁾Number of droplets absorbed at t = 600 seconds.
⁵⁾Polyethylene glycol
⁶⁾The volume ratio of the Glycerol/PEG⁵⁾ (400) mixture: 1:1
⁷⁾The volume ratio of the Glycerol/PEG⁵⁾ (400)/Water mixture: 1:1:2
⁸⁾The volume ratio of the 1,4-butanediol/PEG⁵⁾ (400) mixture: 1:1

It is important to stress that the time measurement 10 seconds given in the table is somewhat arbitrary. In most tests presented in the table above the absorption of the blood droplets 105 were instantaneous i.e. the blood droplets 105 were absorbed within 1-2 seconds.

Thus, the impregnation of the fluid-pervious surface layer 102 leads to a more or less immediate absorption of the blood droplets 105, while a sanitary napkin without impregnation having blood droplets remaining on the fluid-pervious surface layer for a very long time.

However, in the test with the un-impregnated fluid-pervious surface layer a pressure was put on the fluid-pervious surface layer to promote the contact with the underlying absorbent body, and thus speeding up the absorption rate. Still, the absorption rate of the un-impregnated fluid-pervious surface layer was much slower than the absorption rate measured for the impregnated fluid-pervious surface layer 102.

Such remaining fluid results in the surface of the sanitary napkin becoming wet and sticky, which is a disadvantage both during use and when the sanitary napkin shall be replaced.

### EXAMPLE 2

In another embodiment of the invention the fluid-pervious surface layer 102 was impregnated with a mixture containing glycerol and lactic acid. The procedure described in Example 1 for application of the impregnation was repeated for the fluid-pervious surface layer 102 in this Example 2. A similar absorption rate as obtained for the glycerol-impregnated fluid-pervious surface layer 102 in Example 1 was obtained for the glycerol-lactic acid impregnated fluid-pervious surface layer 102. Hence, the lactic acid did not affect the absorbency-rate negatively. However, the lactic acid does offer a good pH control in the urogenital region. A good pH control will counteract bad smells and irritation of the skin or mucous membrane.

In the examples above the impregnation was applied evenly on the absorbent napkin. Of course, it is possible to apply the impregnation only on a portion or portions of the fluid-pervious surface layer of the sanitary napkin, for instance in the wetting region.

The present invention is not limited to the embodiments herein illustrated and described, thus, it is possible to make changes or modifications to the embodiments without departing from the scope of the invention.

An absorbent article according to the invention may comprise further components such as, means for fastening of the article in a pair of panties, shaping elements, barriers, etc.

Although the invention has been described in connection with a sanitary napkin, it is of course also possible to apply the invention to a panty liner or an incontinence protector.

1. A disposable absorbent article to be worn by a user comprising an absorbent body (104) and a cover enclosing the same, which on a first side, facing the user in an in-use position, displays a fluid-pervious surface layer (102) and on a second side, facing away from the user in an in-use position, displays a fluid-impervious surface layer (103), wherein the fluid-pervious surface layer (102) comprises an impregnation comprising at least one hydrophilic organic solvent having a low vapour pressure at room temperature.

2. The absorbent article according to claim 1, wherein the organic solvent has a vapour pressure less than or equal to 1 mm Hg at 40° C.

3. The absorbent article according to claim 1, wherein the organic solvent has a high oxygen content.

4. The absorbent article according to claim 1, wherein the hydrophilic organic solvent is selected from the group consisting of alcohols, ethers, glycol ethers, polyether alcohols, and a mixture thereof.

5. The absorbent article according to claim 4, wherein the hydrophilic organic solvent is glycerol.

6. The absorbent article according to claim 1, wherein the hydrophilic organic solvent is ethylene glycol, polyethylene glycol, 2-ethoxyethanol, 1,1′-oxybis(2-ethoxy) ethane, 1,1′-oxybis[2-methoxyethane], 2,2′-oxybisethanol, 2-methyl-2,4-pentane diol, propylene glycol, triethylene glycol, 1,3-butylene glycol, 2,3-butylene glycol, tetraglyme, or a mixture thereof.

7. The absorbent article according to claim 1, wherein the impregnation further comprises an acid.

8. The absorbent article according to claim 7, wherein the acid is lactic acid.

9. The absorbent article according to claim 3, wherein the oxygen content is at least 30%.
